(19) United States
(12) Patent Application Publication
(10) Pub. No.: US 2012/0232409 A1
(43) Pub. Date: Sep. 13, 2012

(54) SYSTEM AND METHOD FOR RENAL ARTERY OCCLUSION DURING RENAL DENERVATION THERAPY

(76) Inventors: Jeffrey E. Stahmann, Ramsey, MN (US); Allan C. Shuros, St. Paul, MN (US)

(21) Appl. No.: 13/327,161

(22) Filed: Dec. 15, 2011

Related U.S. Application Data
(60) Provisional application No. 61/423,437, filed on Dec. 15, 2010.

(51) Int. Cl. A61B 5/0205; A61B 5/0215; A61B 5/027; A61B 5/026; A61B 5/021

(52) U.S. Cl. 600/483; 600/504; 600/485; 600/505; 600/486

(57) ABSTRACT

A catheter includes a flexible shaft having a length sufficient to access a patient's renal artery relative to a percutaneous access location. A treatment arrangement is provided at a distal end of the shaft and configured for deployment in the renal artery. The treatment arrangement includes an ablation arrangement configured to deliver renal denervation therapy. An occlusion arrangement is configured for deployment in the renal artery and for altering blood flow through the renal artery during or subsequent to renal denervation therapy delivery. A monitoring unit is configured for monitoring for a change in one or more physiologic parameters influenced by the renal denervation therapy. The monitoring unit is configured to produce data useful in assessing effectiveness of the renal denervation therapy based on the physiologic parameter monitoring.

### RELATED PATENT DOCUMENTS

This application claims the benefit of Provisional Patent Application Ser. No. 61/423,437 filed Dec. 15, 2010, to which priority is claimed pursuant to 35 U.S.C. §119(e) and which are hereby incorporated herein by reference.

### SUMMARY

Embodiments of the disclosure are directed to apparatuses and methods for altering blood flow through one or more renal arteries prior to and during and/or subsequent to delivery of a renal denervation therapy, and monitoring for a change in one or more physiologic parameters influenced by the altered renal artery blood flow for assessing renal denervation therapy effectiveness. According to various embodiments, apparatuses include a catheter comprising a flexible shaft having a proximal end, a distal end, and a length sufficient to access a patient's renal artery relative to a percutaneous access location. An ablation arrangement is provided at a distal end of the shaft and configured for deployment in the renal artery. The ablation arrangement is configured to deliver renal denervation therapy, such as for ablating perivascular renal nerves adjacent an outer wall of the renal artery. An occlusion arrangement is configured for deployment in the renal artery and for altering blood flow through the renal artery. A monitoring unit is configured for monitoring for a change in one or more physiologic parameters influenced by the renal denervation therapy. The monitoring unit is configured to produce data useful in assessing effectiveness of the renal denervation therapy based on physiologic parameter monitoring prior and during/subsequent to delivery of renal denervation therapy.

In accordance with other embodiments, methods involve delivering renal denervation therapy to a first renal artery, and altering blood flow through one or both of the first renal artery and a second renal artery during or subsequent to renal denervation therapy delivery. Methods also involve monitoring for a change in one or more physiologic parameters influenced by the altered renal artery blood flow, and assessing effectiveness of the renal denervation therapy based on the physiologic parameter monitoring.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIGS. 4 and 5 are flow charts illustrating various processes for assessing the efficacy of renal denervation therapy in accordance with various embodiments;

FIG. 6 shows the hemodynamic response to a reduction in renal artery flow in a dog model;

FIGS. 7A and 7B show systems for ablating innervated renal tissue and assessing the efficacy of renal denervation therapy in accordance with various embodiments; and

FIGS. 8-19 illustrate a multiplicity of combined ablation and occlusion arrangements in accordance with various embodiments.

### DETAILED DESCRIPTION

Embodiments of the disclosure are directed to systems and methods for assessing the effectiveness of renal nerve ablation. The assessment involves measuring the functional status of the renal portion of a patient's autonomic nervous system via occlusion of one or both of the renal arteries during and/or after a renal nerve ablation procedure. Monitoring a physiologic response or lack of response to renal artery occlusion is used to assess the efficacy of renal denervation.

Studies show that renal nerve ablation can chronically reduce blood pressure in hypertensive patients. Pre-clinical studies suggest that renal sympathetic nerve denervation improves outcomes in heart failure (HF) models. Chronic benefit of the ablation procedure is dependent on relatively complete denervation of the renal nerves. There is currently no reliable and accurate method for determining if the renal nerves have been successfully ablated during a renal ablation procedure.

It has been found that an abrupt and significant reduction in renal blood flow in a patient with a functioning autonomic nervous system results in an abrupt and significant increase in systemic blood pressure due to afferent kidney-to-brain nerve activity. However, if the renal portion of the patient's autonomic nervous system is disrupted due to the ablation procedure, an abrupt and significant reduction in renal blood flow should result in minimal (e.g., negligible) change in systemic blood pressure. Embodiments of the disclosure are directed to systems and methods that provide for control of renal blood flow and measurement of systemic blood pressure and/or other physiologic parameter(s) modulated by changes in renal blood flow, and for assessing the efficacy of the renal nerve ablation during and/or after the ablation procedure.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the body's “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

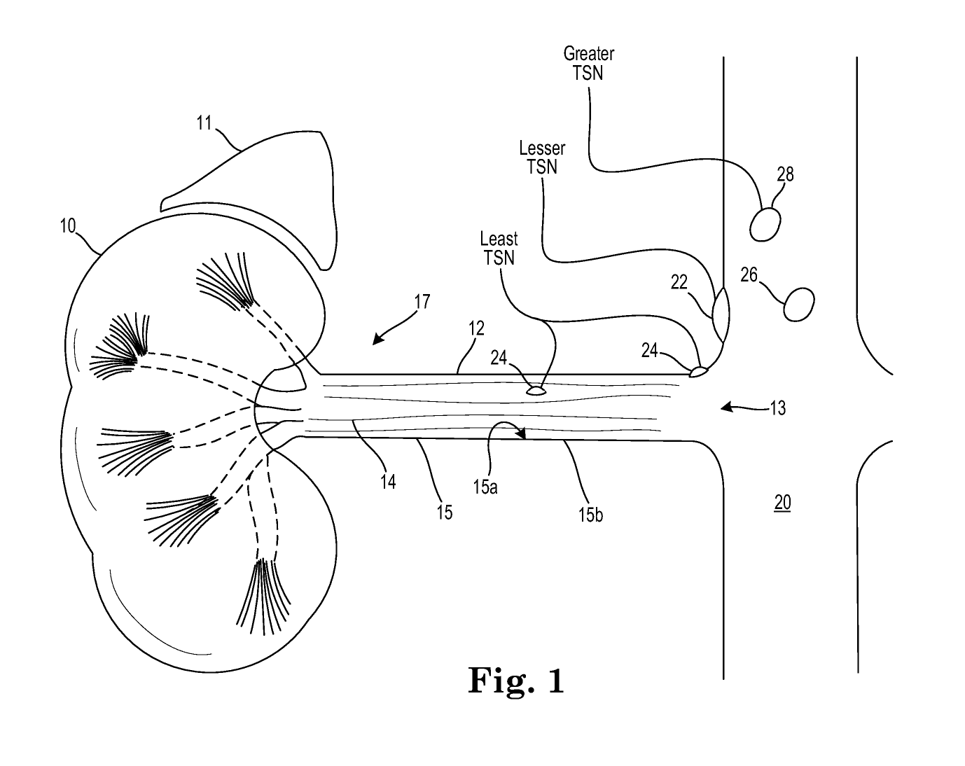

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

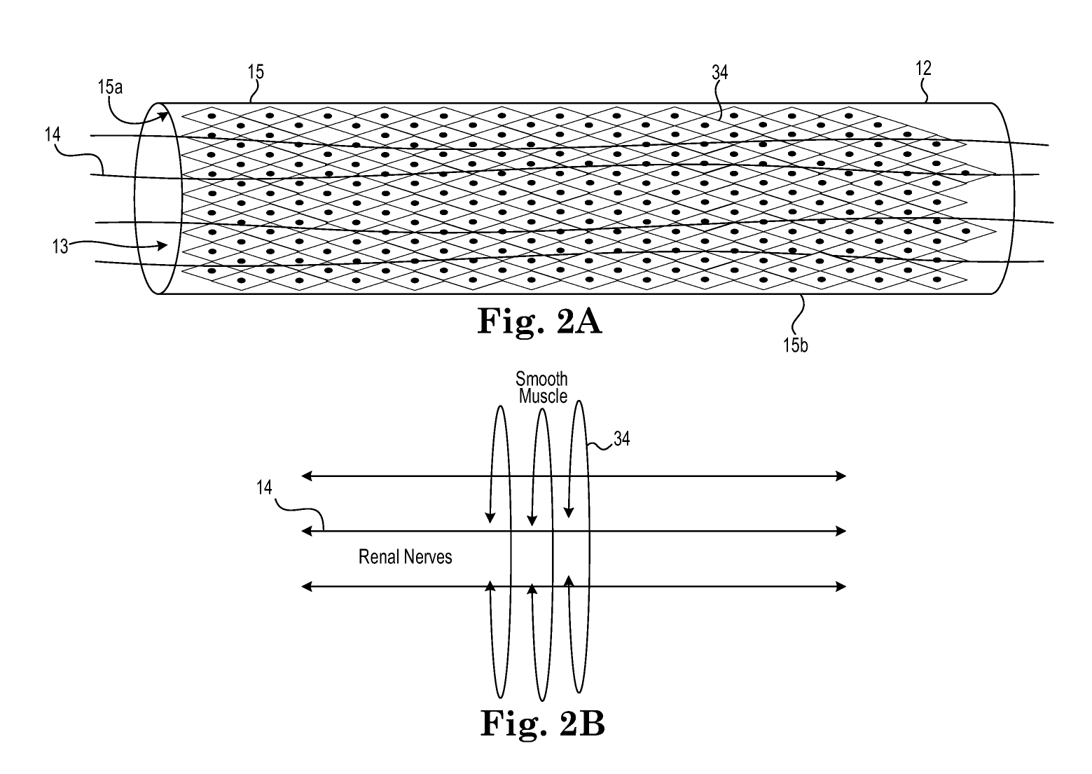

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

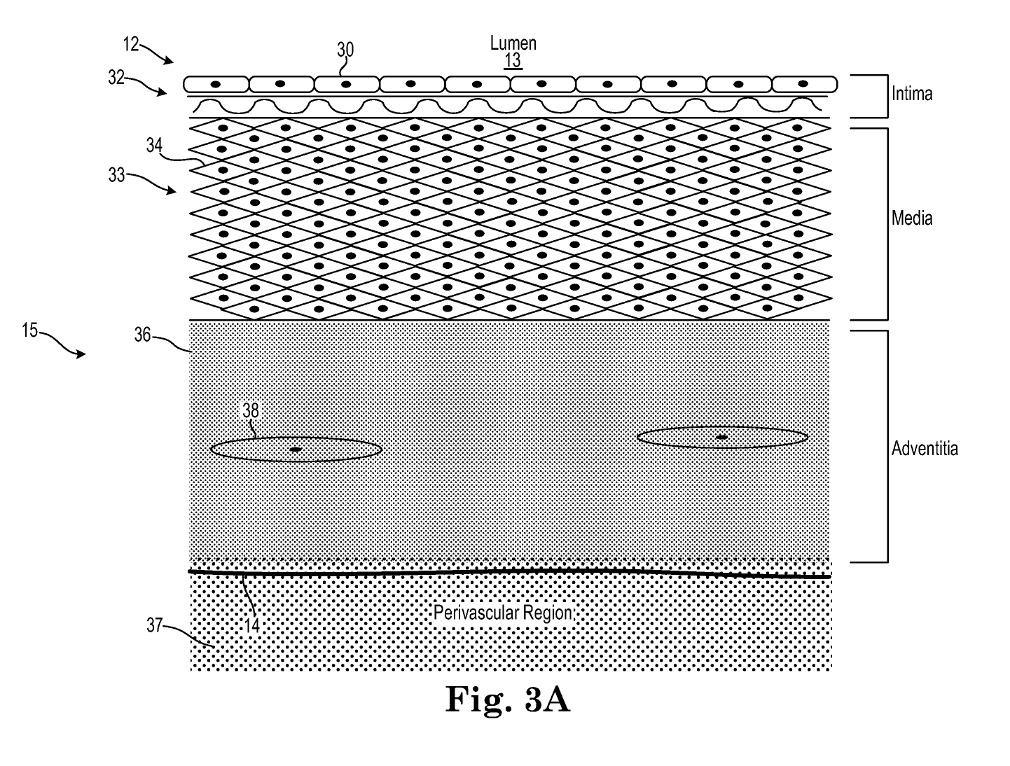

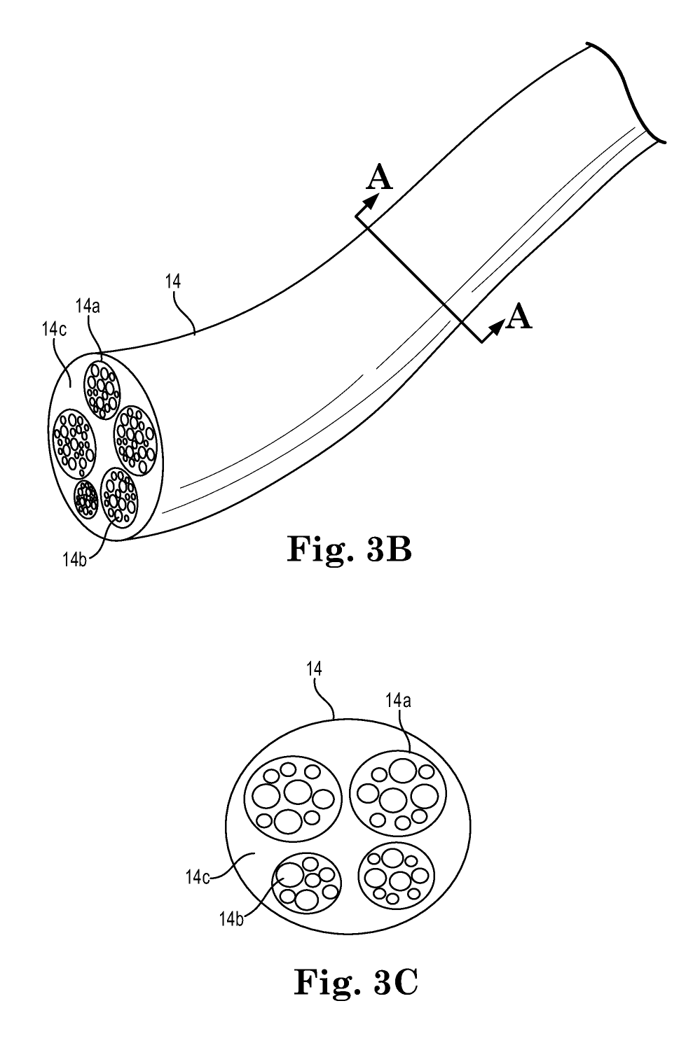

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b is preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

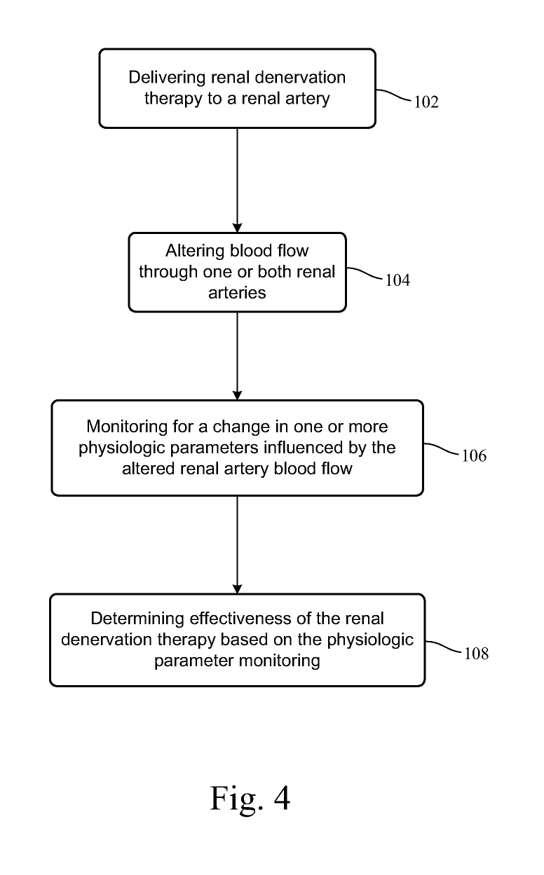

FIG. 4 illustrates various processes for assessing the efficacy of renal denervation therapy in accordance with various embodiments. Assessing renal denervation therapy is preferably performed in real-time during renal ablation therapy delivery. The representative method illustrated in FIG. 4 involves delivering 102 renal denervation therapy to one of the patient's renal arteries. Renal denervation therapy cause transient or permanent loss of nerve function and may be delivered using various techniques and agents. Useful ablative agents in accordance with various embodiments include the following: electrical energy; optical energy for thermally ablating the renal nerves proximate the target sites; optical energy for forming micro-bubbles within renal nerve tissue proximate the target sites to mechanically disrupt nerve fibers and ganglia included within the renal nerve tissue upon implosion or explosion; acoustic energy for thermally ablating the renal nerves proximate the target sites; acoustic energy for forming micro-bubbles within renal nerve tissue proximate the target sites to mechanically disrupt nerve fibers and ganglia included within the renal nerve tissue upon implosion or explosion; mechanical loading or compressive force; cryothermal energy; thermal energy sufficient to cause coagulation, denaturation or necrosis; a neurotoxin or a venom; and an induced pH change sufficient to cause necrosis.

During and/or following renal denervation therapy delivery, blood flow through one or both of the patient's renal arteries is altered 104 in a manner that can elicit a physiologic response. For example, the renal artery or arteries can be occluded so as to abruptly and significantly reduce blood flow through the renal artery or arteries. Occlusion of a renal artery can be partial or complete, and the degree of occlusion may be the same or different with respect to the two renal arteries. The physiologic parameter data acquired prior to renal denervation that show an abrupt change due to a reduction in renal blood flow is preferably stored and later used as pre-denervation baseline data. This data shows that the patient has a functioning autonomic nervous system due to afferent kidney-to-brain nerve activity.

Monitoring 106, for example, may involve monitoring for an abrupt change in the one or more physiologic parameters that exceeds a threshold indicative of an acute response to the renal artery blood flow alteration. The effectiveness of the renal denervation therapy is determined 108 based on the physiologic parameter monitoring. For example, detecting or lack of detecting a significant change in systemic blood pressure responsive to the altered renal artery blood flow provides an indication of renal denervation therapy effectiveness. Based on the therapy effectiveness assessment, renal denervation therapy can be terminated (i.e., therapy was effective) or continued (i.e., therapy was ineffective or partially effective).

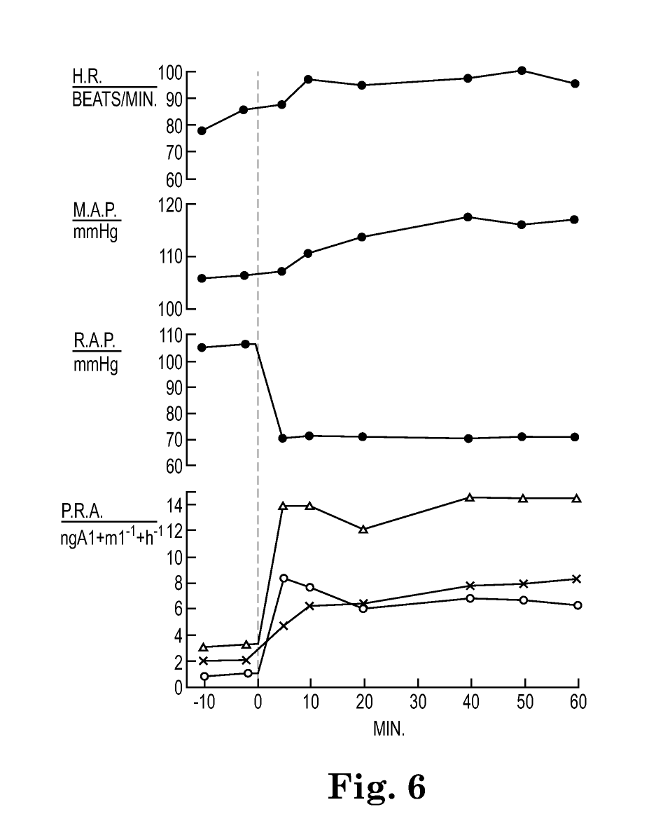

A number of different physiologic parameters can be monitored individually or in combination. A representative list of useful physiologic parameters that can be monitored for assessing completeness of renal denervation therapy include: systemic blood pressure; cardiac chamber (e.g. left ventricular) blood pressure; pulmonary blood pressure; renal blood flow; a neuro-hormonal level; a renin level; arterial constriction or dilation; heart rate; cardiac output; and cardiac stroke volume. An abrupt acute change in a particular physiologic parameter in the context of various embodiments is understood to be a readily noticeable or significant change in the state of the parameter in response to an induced alteration in renal artery blood flow (see., e.g., the data plotted in FIG. 6), which is indicative of a functioning autonomic nervous system due to afferent kidney-to-brain nerve activity. A lack of, or negligible change in, a particular physiologic parameter is understood to be indicative of a non-functioning autonomic nervous system due to effective renal denervation therapy.

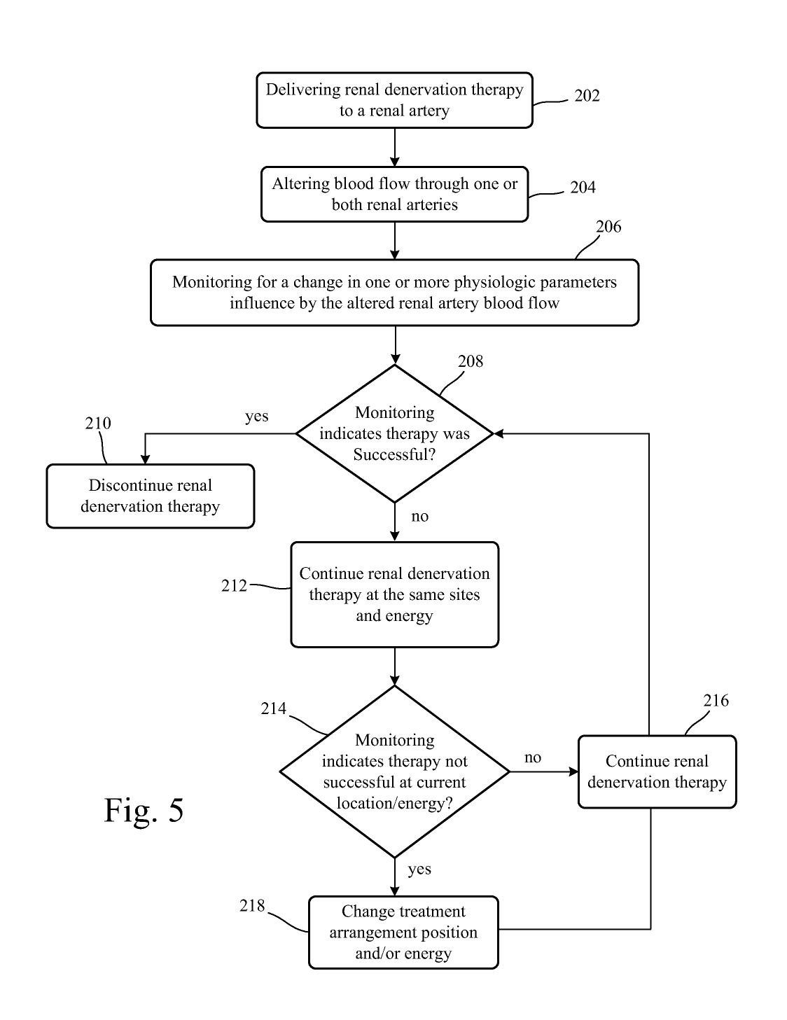

FIG. 5 illustrates various processes for assessing the efficacy of renal denervation therapy in accordance with various embodiments. According to the embodiment illustrated in FIG. 5, renal denervation therapy is delivered 202 to one of the patient's renal arteries. Blood flow through one or both of the patient's renal arteries is altered 204 during and/or subsequent to renal denervation therapy delivery. The method further involves monitoring 206 for a change in one or more physiologic parameters influenced by the altered renal artery blood flow during and/or subsequent to renal denervation therapy delivery. If the monitored physiologic parameter(s) indicate that the renal denervation therapy was successful 208, the therapy is discontinued 210. The processes of FIG. 5 may be repeated for the patient's other renal artery in this case. If, however, the monitored physiologic parameter(s) indicate that the therapy has not achieved success, renal denervation therapy is continued 212 at the same site(s) and energy for a longer duration.

If further monitoring 214 of the physiologic parameter(s) continue to indicate that the therapy is not successful at the current sites(s) and energy, one or both of the position of the ablation arrangement and energy is adjusted. Renal denervation therapy continues 216 after making the appropriate adjustments. If the monitored physiologic parameter(s) indicate that renal denervation therapy is successful after the ablation arrangement position and/or energy adjustment, renal denervation therapy is discontinued 210. The processes shown in FIG. 5 are repeated until such time as the monitored physiologic parameter(s) indicate that renal denervation therapy was successful or unsuccessful for each treatment site.

FIG. 6 shows the hemodynamic response (M.A.P., H.R.) to a reduction in renal artery flow in a dog model. More particularly, FIG. 6 shows the effect of a reduction of mean renal artery pressure on plasma renin activity (P.R.A). It can be seen that time=0, the renal artery pressure was reduced to 70 mm Hg. Thereafter, renal artery pressure was kept constant at this level for 1 hour. In FIG. 6, H.R. refers to heart rate; M.A.P. refers to mean aortic blood pressure; R.A.P. refers to mean renal artery pressure; Δ is data for renal-venous P.R.A.; x is data for arterial P.R.A.; and O is data for renal-venous-arterial P.R.A.-difference. The data plotted in FIG. 6 clearly show an abrupt change in R.A.P and P.R.A. in response to altering (e.g., occluding) blood flow through the renal artery prior to renal denervation. This data, when acquired for a patient prior to a renal denervation procedure, can serve as pre-denervation baseline data. During and/or following a renal denervation procedure, the data shown in FIG. 6 can be re-acquired and compared to the pre-denervation baseline data. If the renal portion of the patient's autonomic nervous system has been disrupted due to the ablation procedure, only a minimal (e.g., negligible) change in R.A.P. and P.R.A. should be detected, indicating that the renal denervation procedure was successful.

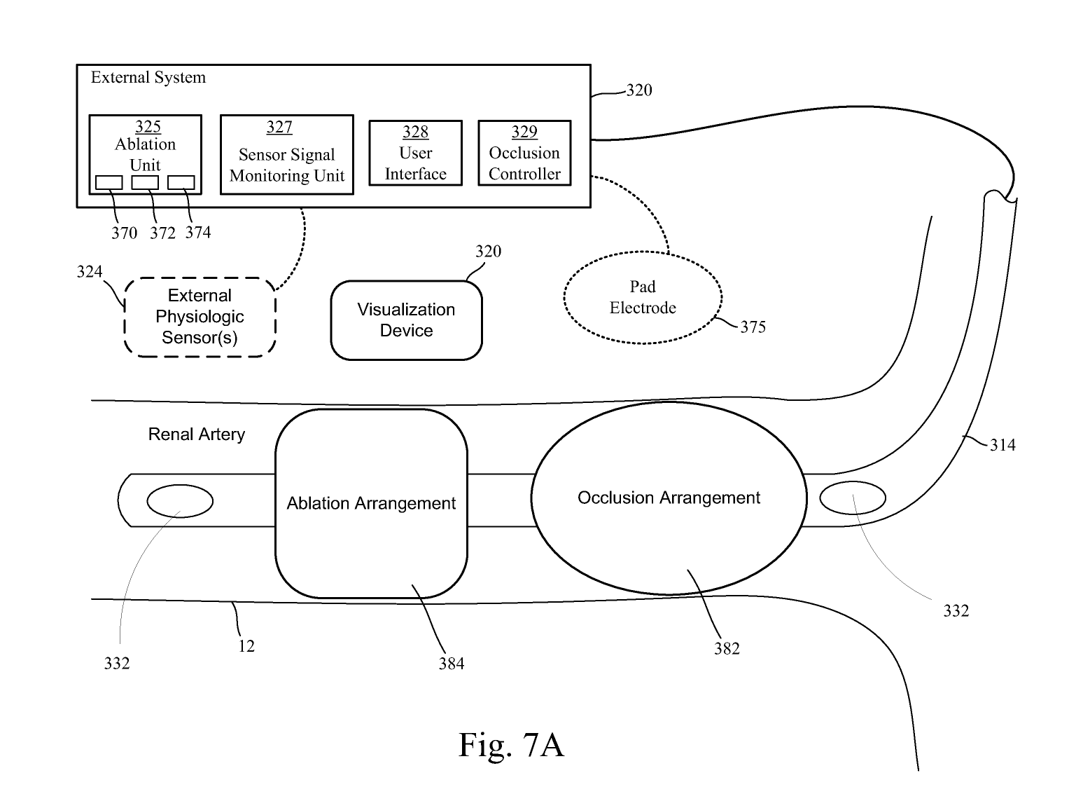

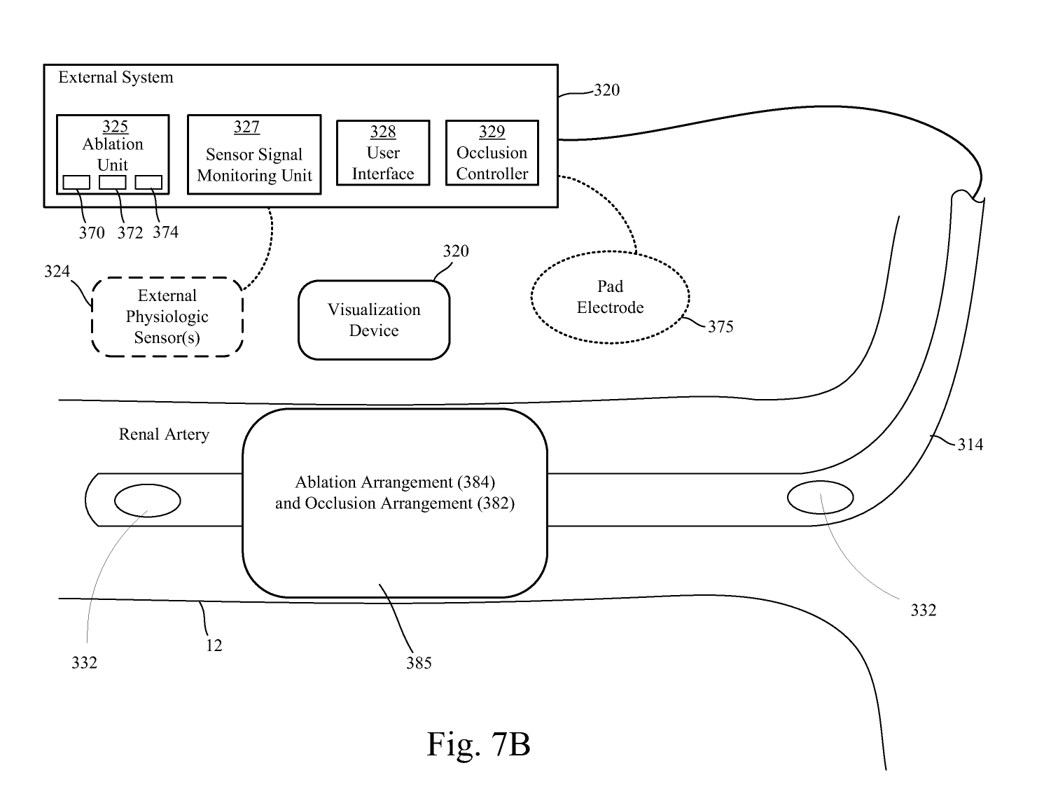

Referring now to FIG. 7A, there is illustrated a system for ablating renal nerves and occluding a renal artery to elicit a physiologic response for evaluating the efficacy of renal denervation therapy in accordance with various embodiments. The system shown in FIG. 7A includes an external system 320 coupled to a catheter having a flexible shaft 314 with a length sufficient to access the patient's renal artery 12 relative to a percutaneous access location. An ablation arrangement 384 is provided at a distal end of the shaft 314 and configured for deployment in the renal artery 12. The ablation arrangement 384 is configured to deliver renal denervation therapy. The ablation arrangement 384 can include one or more ablation devices configured to deliver the same or different denervation agents. A catheter delivery system may be employed to facilitate delivery of the ablation arrangement 384 and occlusion arrangement 382 into the renal artery 12. A delivery sheath and/or a guide catheter, for example, may be used.

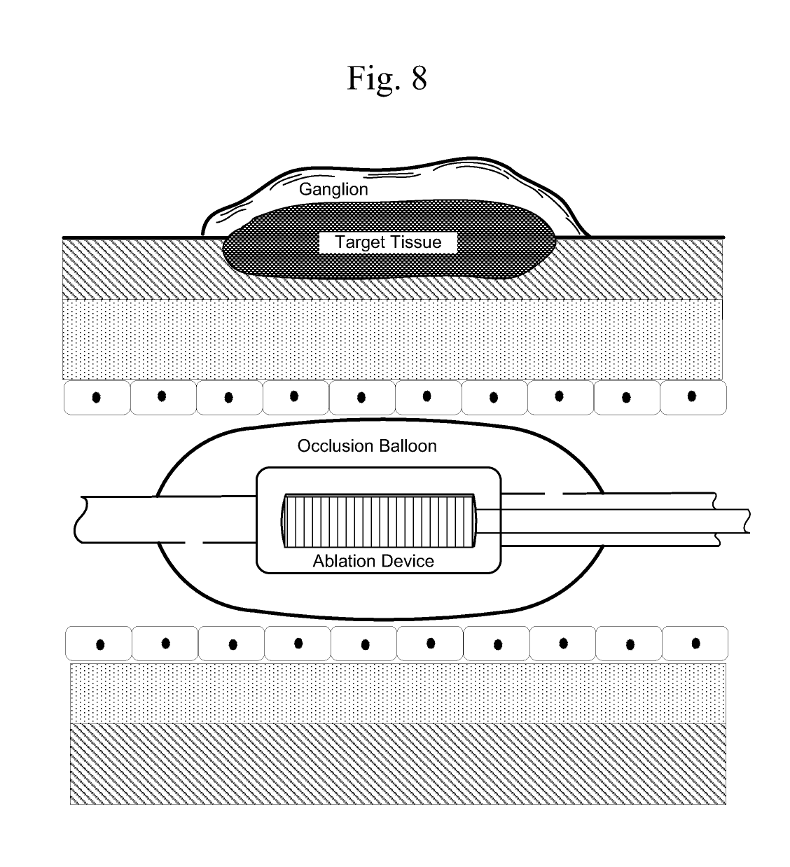

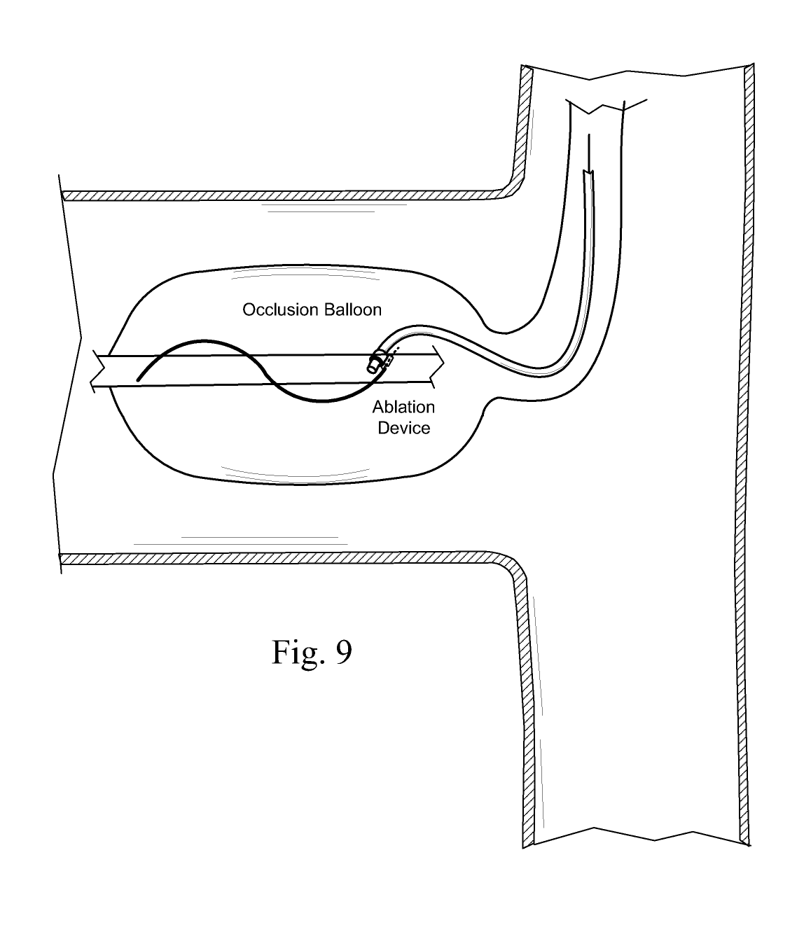

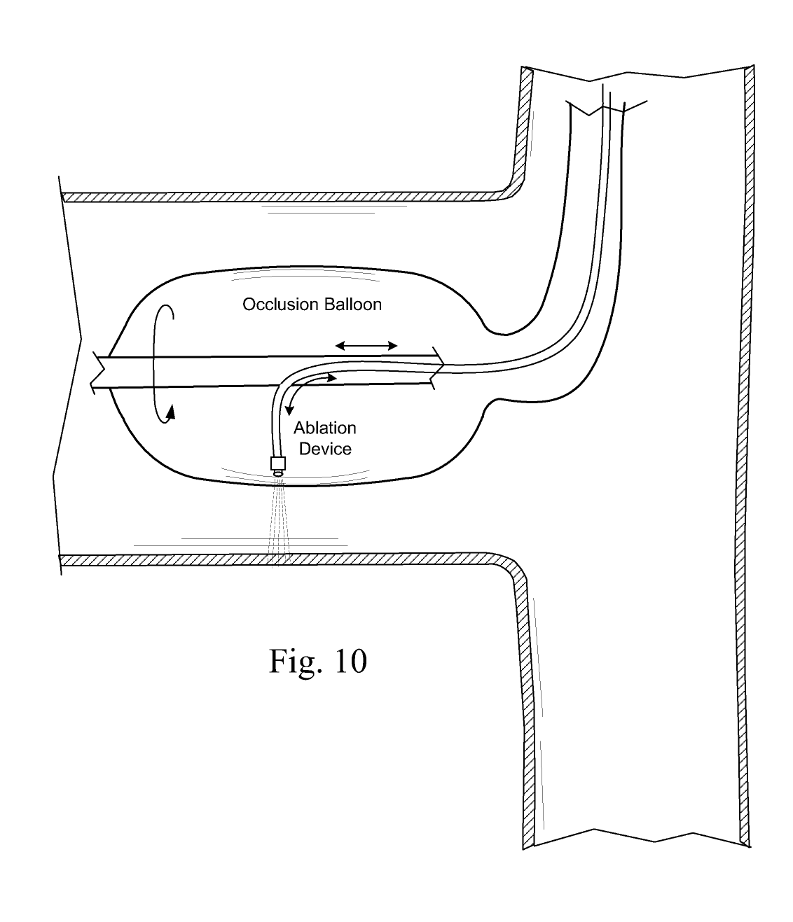

The ablation arrangement 384 may be configured to deliver one or more of the renal denervation agents listed above and/or those disclosed in the following commonly owned, co-pending US published and pending patent applications identified as: 20110257523; 20110257641; 20110263921; 20110264086; and 20110264116; and U.S. patent application Ser. No. 13/157,844 filed Jun. 10, 2011; Ser. No. 13/188,677 filed Jul. 22, 2011; Ser. No. 13/193,338 filed Jul. 28, 2011; Ser. No. 13/184,677 filed Jul. 18, 2011; Ser. No. 13/228,233 filed Sep. 8, 2011; Ser. No. 13/281,962 filed Oct. 26, 2011; Ser. No. 13/243,114 filed Sep. 23, 2011; Ser. No. 13/243,724 filed Sep. 23, 2011; Ser. No. 13/295,185 filed Nov. 14, 2011; Ser. No. 13/243,729 filed Sep. 23, 2011; and Ser. No. 13/299,932 filed Nov. 18, 2011; each of which is incorporated herein by reference. A representative set of renal denervation device illustrations disclosed in these commonly owned co-pending applications are provided as FIGS. 8-19. It is understood that various embodiments may incorporate one or more of the systems, devices, sensors, features and/or functions disclosed in these commonly-owned co-pending U.S. published and pending patent applications.

With continued reference to FIG. 7A, an occlusion arrangement 382 is configured for deployment in the renal artery 12 and controllable for altering blood flow through the renal artery 12 prior, during and/or subsequent to renal denervation therapy delivery. The occlusion arrangement 382 is situated proximal of the ablation arrangement 384 on the catheter shaft 314. In some embodiments, such as that shown in FIG. 7A, the occlusion arrangement 382 is supported by the catheter shaft 314 that also supports the ablation arrangement 384. In other embodiments, the occlusion arrangement is provided on a catheter separate from that which supports the ablation arrangement 384.

The occlusion arrangement 382 is preferably transformable between a low-profile introduction configuration and a larger profile deployed configuration. The occlusion arrangement 382 may incorporate a balloon or other expandable/contractible structure (wire or mesh structure covered by a polymer) that is dimensioned to occlude arterial blood flow when deployed in the renal artery 12. The occlusion arrangement 382 is preferably controllable between a low-profile introduction configuration, a fully occluding deployed configuration, and a partially occluding deployed configuration. In some embodiments, multiple occlusion catheters can be employed to provide for simultaneous occlusion of multiple renal arteries.

An occlusion controller 329 of the external system 320 provides for controlled expansion and contraction/collapsing of the occlusion arrangement 382. In embodiments that employ an occlusion balloon, the occlusion controller 329 controls inflation and deflation of an occlusion balloon for transforming the occlusion arrangement 382 between low-profile introduction and larger profile deployed configurations. In embodiments that employ an expandable/collapsible polymer covered wire or mesh structure, for example, the occlusion controller 329 can include a push/pull actuation arrangement that mechanically causes expansion and collapsing of the occlusion arrangement 382. In further embodiments, a self-expanding occlusion structure can be employed, such as a Nitinol or other shape-memory or spring-like structure. The self-expanding occlusion structure can be transformed between low-profile introduction and larger profile deployed configurations by advancing the occlusion structure constrained within a lumen of a delivery sheath or guide catheter beyond the distal tip of the delivery sheath or guide catheter.

In various embodiments that provide for partial renal artery occlusion, the occlusion arrangement 382 may incorporate a profusion feature, such as longitudinal flutes that allow for passage of a specified small volume of arterial blood when the occlusion arrangement 382 is in its deployed configuration within the renal artery 12. In some embodiments, the shaft 314 may incorporate diversion ports located proximally and distally of the occlusion arrangement 382, allowing for a specified volume of arterial blood to flow through a lumen within the shaft 314 and fluidly coupled to the proximal and distal diversion ports.

In the embodiment shown in FIG. 7A, the catheter shaft 314 supports one or more physiologic sensors 332, each of which has a response that is moderated by changes in renal function. The physiologic sensors 332 can be of the same or different type. In some embodiments, implantable physiologic sensors 332 are employed. Implantable physiologic sensors 332 may be located on shaft 314 or elsewhere in the body. In other embodiments, an external physiologic sensors 324 are used. In further embodiments, a combination of implantable and external physiologic sensors 332, 324 are employed. Useful physiologic sensors 332, 324 includes the following non-limiting representative devices: a systemic blood pressure sensor; a left ventricular blood pressure sensor, a pulmonary blood pressure sensor, a renal blood flow sensor; a neuro-hormonal sensor; a renin sensor; an arterial constriction or dilation sensor, a heart rate sensor, and a cardiac stroke volume sensor. In some embodiments, blood flow sensors 332 located distal and proximal to the occlusion arrangement 382 can be employed.

The external system 320 includes a user interface 328 that provides visualization and/or monitoring of various information concerning the location of the ablation catheter and status of the occlusion mechanism within the renal artery or arteries. The external system 320 may be coupled to a visualization device 320, which may include an MRI, ultrasound, or fluoroscopy system or device. The user interface 328 preferably provides for viewing the occlusion status of the renal artery via renal blood flow (or other parameter), together with visualization of the occlusion mechanism. The user interface 328 also presents monitoring data acquired by a sensor signal monitoring unit 327 for one or a number of physiologic parameters that are moderated by changes in renal function due to ablation of renal nerves.

According to some embodiments that include an electrode or electrode array as part of the ablation arrangement 384, high-frequency AC energy is used ablate renal nerve tissue passing between the ablation electrode/array and an external pad electrode 375 in a unipolar RF ablation mode. The pad electrode 375 can be placed on a portion of the patient's skin proximate the renal arteries, and serves as an external electrode when operating in a unipolar mode. In some embodiments, a multiplicity of ablation electrodes or sets of electrodes can be configured to deliver RF energy to renal nerve tissue in a bipolar ablation mode, in which case an external pad electrode 375 is not needed.

An ablation unit 325 and the ablation arrangement 384 cooperate to ablate renal nerve sites. The ablation unit 325 and the ablation arrangement 384 cooperate to deliver an ablative agent or agents to the target renal artery sites. Various types of ablative agents can be used to ablate the target renal artery sites, non-limiting examples of which include those described previously hereinabove. According to some embodiments, the ablation unit 325 can include a control unit 370, energy delivery protocols 372, and a sensor unit 374. The control unit 370 is configured to control operation of the ablation unit 325, including implementation of one or more energy delivery protocols 372. The sensor unit 374 is coupled to one or more sensors which may be positioned at the ablation arrangement 384 or elsewhere on the catheter shaft 314, another catheter, or an external system, for sensing and monitoring one or more parameters during renal nerve ablation. Useful sensors include temperature, impedance, voltage, acoustic, and tissue stiffness (elasticity) sensors, for example. In some embodiments, sensor data acquired by the sensor unit 374 during ablation allows the ablation unit 325 to control the ablation procedure in an automatic or semi-automatic mode.

The system shown in FIG. 7B is the same as that shown in FIG. 7A, except that the occlusion arrangement 382 and the ablation arrangement 384 are configured as a combined apparatus 385. In some embodiments, the ablation arrangement 384 is provided on the catheter shaft 314 and the occlusion arrangement 382 encompasses all or at least a portion of the ablation arrangement 384. In other embodiments, the ablation arrangement 384 is supported at least in part by the occlusion arrangement 382. Transitioning the occlusion arrangement 382 from a low-profile introduction configuration to a larger profile deployed configuration moves at least a portion of the ablation arrangement 384 into contact or near-contact with the inner wall of the renal artery 12.

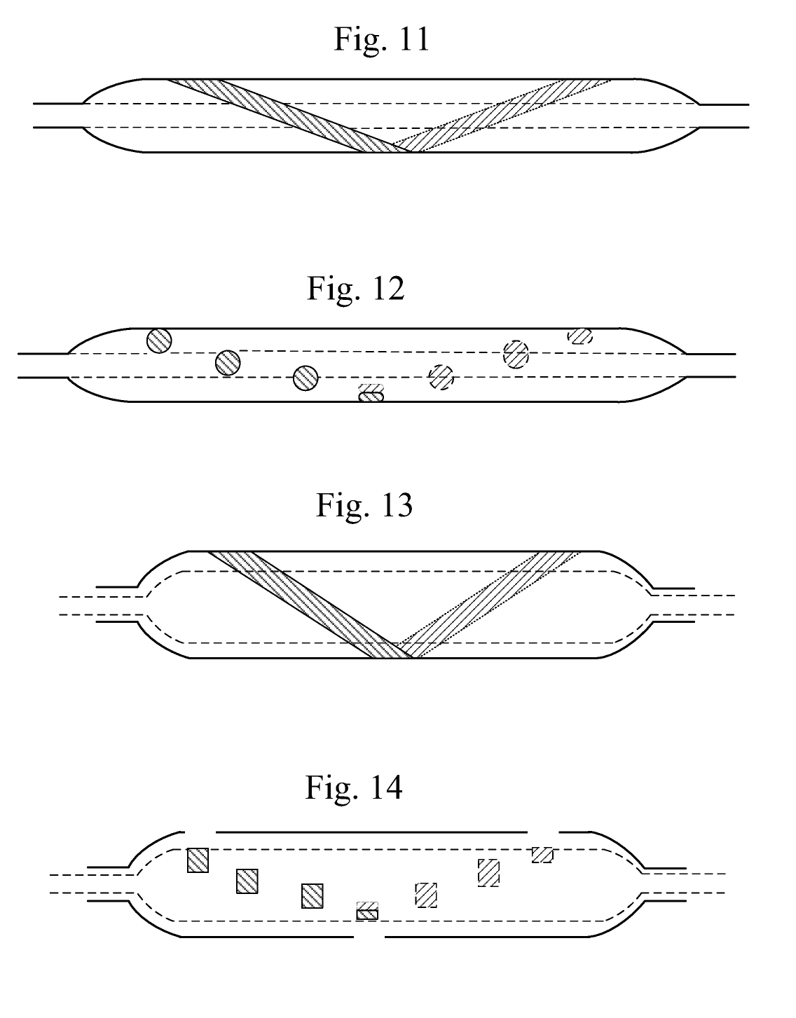

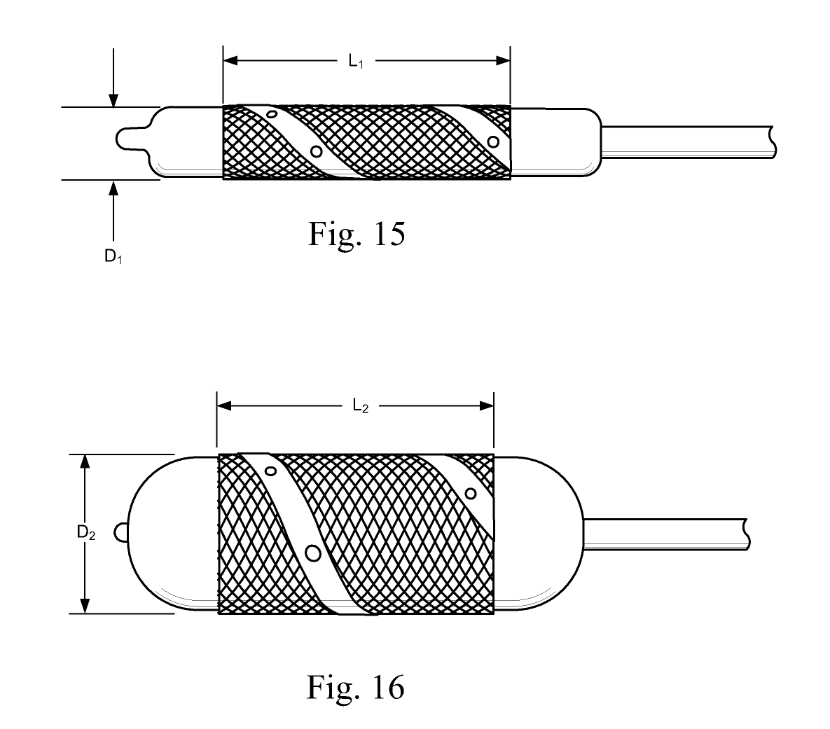

FIGS. 8-19 illustrate a multiplicity of combined ablation and occlusion arrangements 385 in accordance with various embodiments. The embodiments shown in FIGS. 8-19 are disclosed and described in various commonly owned, co-pending US patent applications identified hereinabove. FIGS. 8-10 show different configurations of an ablation device within an occlusion balloon. The ablation device shown in FIGS. 8-10 can include an ultrasound (e.g., HIFU) transducer or an optical (e.g., laser) transducer mounted on the catheter shaft and encompassed by an occlusion balloon. FIGS. 11-14 show different configurations of cryothermal occlusion balloons with patterning features that facilitate creation of lesions having a predetermined shape (e.g., a spiral or helix). FIGS. 15 and 16 show an occlusion balloon in a low-profile introduction configuration (FIG. 15) and a deployed configuration (FIG. 16). The balloon supports an expandable braid that incorporates a patterned RF electrode arrangement.

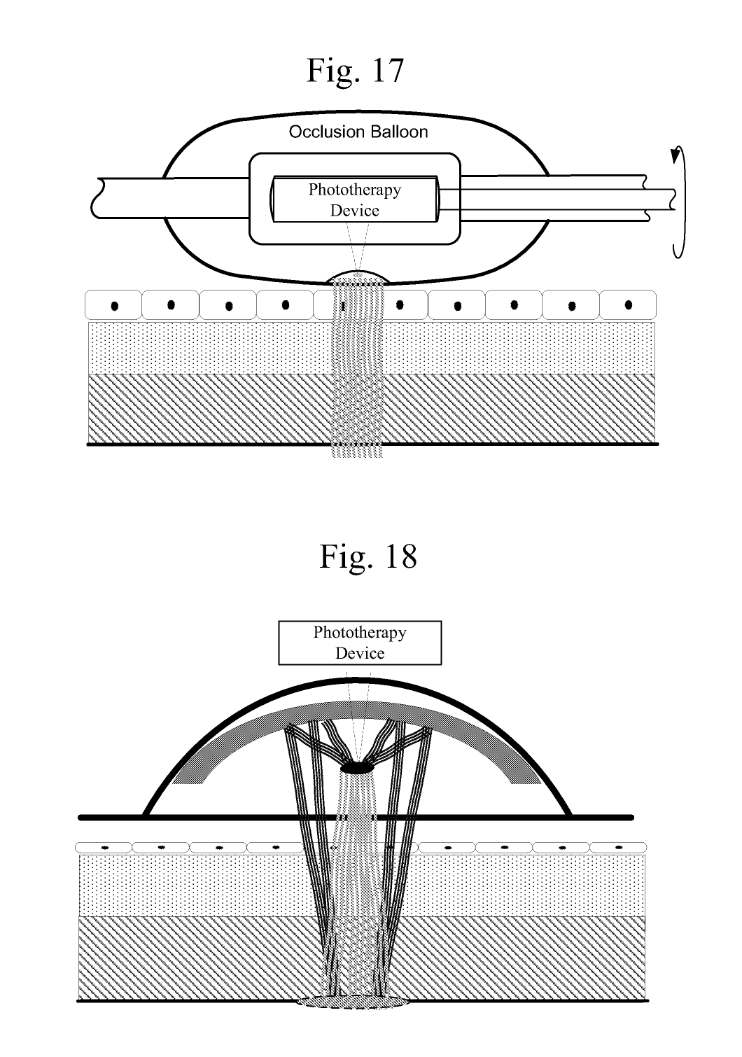

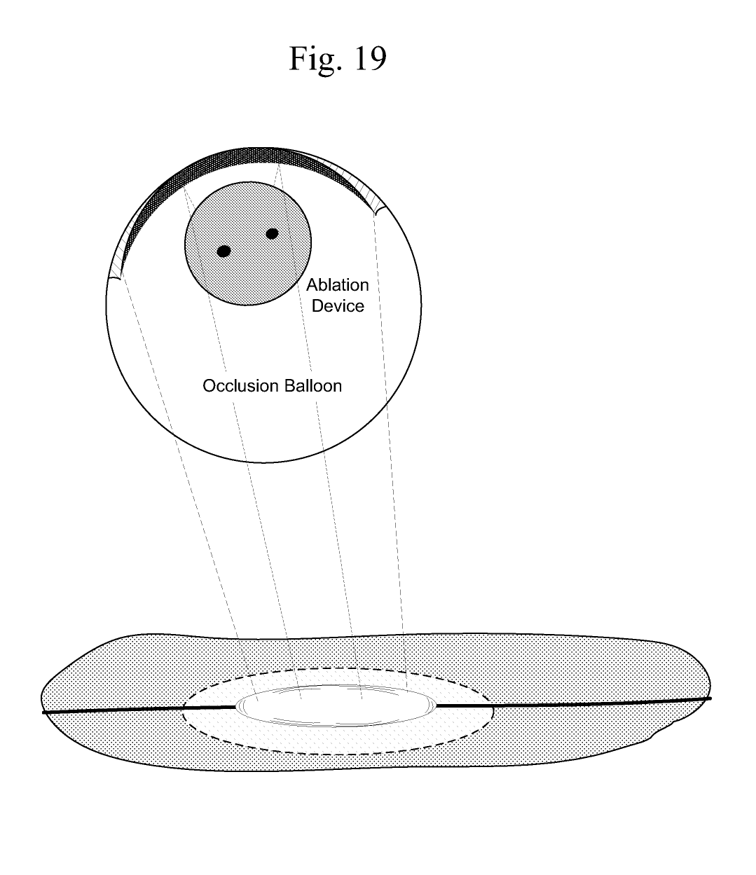

FIG. 17 shows a phototherapy device supported on the catheter shaft and encompassed by an occlusion balloon. A small pocket of fluid integrated into the balloon (best seen in FIG. 18) receives optical energy of sufficient intensity to create a cavitation bubble in the contained fluid, and to cause the bubble to burst, thereby generating an acoustic shock wave which is directed to innervated renal tissue. FIG. 19 shows a light source supported by a catheter shaft (not shown) and encompassed by an occlusion balloon. The light source preferably produces white light of sufficient intensity to ablate renal nerves and ganglia. Various other combinations of ablation arrangements and occlusion structures are contemplated.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. A method, comprising:
delivering renal denervation therapy to a first renal artery;
altering blood flow through one or both of the first renal artery and a second renal artery during or subsequent to renal denervation therapy delivery;
monitoring for a change in one or more physiologic parameters influenced by the altered renal artery blood flow; and
assessing effectiveness of the renal denervation therapy based on the physiologic parameter monitoring.

2. The method of claim 1, wherein altering blood flow comprises altering blood flow through the first and second renal arteries during or subsequent to renal denervation therapy delivery.

3. The method of claim 1, wherein altering blood flow comprises fully or partially occluding renal artery blood flow temporarily.

4. The method of claim 1, wherein assessing effectiveness of the renal denervation therapy is effected during a renal denervation therapy procedure.

5. The method of claim 1, wherein monitoring for the change in one or more physiologic parameters comprises:
monitoring, prior to delivery of renal denervation therapy, for an abrupt change in the one or more physiologic parameters indicative of an acute response to the altered renal artery blood flow; and
monitoring, during or subsequent to delivery of renal denervation therapy, for an abrupt change in the one or more physiologic parameters indicative of an acute response to the altered renal artery blood flow, wherein an absence of or a negligible change in the one or more physiologic parameters during or subsequent to delivery of renal denervation therapy indicates successful delivery of renal denervation therapy.

6. The method of claim 1, comprising one or both of visualizing the first renal artery and monitoring renal artery blood flow to confirm the alteration of renal artery blood flow.

7. The method of claim 1, comprising controlling renal denervation therapy delivery in response to the therapy effectiveness assessment.

8. The method of claim 1, comprising continuing renal denervation therapy delivery in response to an unsuccessful therapy effectiveness assessment.

9. The method of claim 1, wherein the change in one or more physiologic parameters is monitored within the first renal artery.

10. The method of claim 1, wherein the change in one or more physiologic parameters is monitored patient externally.

11. The method of claim 1, wherein the one or more physiologic parameters comprises one or more of systemic blood pressure, left ventricular blood pressure, pulmonary blood pressure, and renal blood flow.

12. The method of claim 1, wherein the one or more physiologic parameters comprises one or more of a neuro-hormonal level and a renin level.

13. The method of claim 1, wherein the one or more physiologic parameters comprises one or more of arterial constriction or dilation, heart rate, and cardiac stroke volume.

14. An apparatus, comprising:
a catheter comprising a flexible shaft having a proximal end, a distal end, and a length sufficient to access a patient's renal artery relative to a percutaneous access location;
an ablation arrangement provided at a distal end of the shaft and configured for deployment in the renal artery, the ablation arrangement configured to deliver renal denervation therapy;
an occlusion arrangement configured for deployment in the renal artery and for altering blood flow through the renal artery; and
a monitoring unit configured for monitoring for a change in one or more physiologic parameters influenced by the renal denervation therapy, the monitoring unit configured to produce data useful in assessing effectiveness of the renal denervation therapy based on the physiologic parameter monitoring performed prior to and during or subsequent to delivery of renal denervation therapy.

15. The apparatus according to claim 14, wherein the occlusion arrangement is supported by the catheter shaft.

16. The apparatus according to claim 14, wherein the occlusion arrangement comprises a catheter separate from the catheter supporting the ablation arrangement.

17. The apparatus according to claim 14, wherein the catheter shaft supports one or more physiologic sensors.

18. The apparatus according to claim 14, wherein the monitoring unit is coupled to one or more external physiologic sensors.

19. The apparatus according to claim 14, wherein the monitoring unit comprises one or both of a visualization device and a monitoring device configured to respectively visualize and monitor one or more characteristics of the renal artery during the renal denervation therapy procedure.

20. The apparatus of claim 14, wherein the one or more physiologic parameters comprises one or more of systemic blood pressure, left ventricular blood pressure, pulmonary blood pressure, and renal blood flow.

21. The apparatus of claim 14, wherein the one or more physiologic parameters comprises one or more of a neuro-hormonal level and a renin level.

22. The apparatus of claim 14, wherein the one or more physiologic parameters comprises one or more of arterial constriction or dilation, heart rate, and cardiac stroke volume.
